(19) United States
(12) Patent Application Publication
(10) Pub. No.: US 2002/0048599 A1
(43) Pub. Date: Apr. 25, 2002

(54) Method for increasing tissue perfusion by co-administration of an agent that increases cGMP synthesis and an agent that inhibits cGMP degradation

(76) Inventor: Thomas H. Mueller, Danbury, CT (US)

Correspondence Address: BOEHRINGER INGELHEIM CORPORATION, 900 RIDGEBURY ROAD, P O BOX 368, RIDGEFIELD, CT, 06877, US

(21) Appl. No.: 09/981,335

(22) Filed: Oct. 16, 2001

Related U.S. Application Data
(60) Provisional application No. 60/242,342, filed on Oct. 20, 2000.

(51) Int. Cl. A61K 48/00; A61K 38/05; A61K 31/519; A61K 31/401; A61K 31/225

(52) U.S. Cl. 424/457; 514/44; 514/262.1; 514/19; 514/423; 514/547; 514/171

(57) ABSTRACT

A method for increasing tissue perfusion with blood by the co-administration of an agent that increases cGMP synthesis and an agent that inhibits cGMP degradation in the cells of the blood vessel walls or in blood cells. The method comprises, for example, the co-administration of therapeutically effective amounts of a statin and dipyridamole, especially a timed-release formulation of dipyridamole.

### RELATED APPLICATION

Benefit of U.S. Provisional Application Serial No. 60/242,342, filed on Oct. 20, 2000 is hereby claimed.

### FIELD OF THE INVENTION

The invention relates to a method for increasing tissue perfusion with blood by the coadministration of an agent that increases cGMP synthesis and an agent that inhibits cGMP degradation in the cells of the blood vessel walls or in blood cells.

### BACKGROUND OF THE INVENTION

Decreased tissue perfusion with blood leads to a diversity of functional impairments and overt clinical manifestations of dysfunction. In general, therapeutic regimens that increase the blood flow of organs and tissues can be used to prevent acute and chronic complications of less than optimal tissue perfusion (ischemia).

There is a clear medical need to improve therapies for the prevention and treatment of ischemic disease (arterial thrombosis: myocardial infarction, stroke, peripheral arterial occlusion etc.), improve and maintain optimal organ function by enhanced blood perfusion of tissues (e.g. prevent the impairment in organ function due to atherosclerosis, to prevent and treat nephropathy in diabetics, improve myocardial function in patients with coronary heart disease, cerebral function in elderly or hypertensives, prevent vascular damages in smokers) and prevent cardiovascular death.

It is known that shear forces and various mediators (circulating in blood or locally released by activated platelets and other blood cells) including acetylcholine, histamine, vasopressin, norepinephrine, bradykinin, ADP, serotonin, endothelin and thrombin trigger the synthesis of nitric oxide in endothelial cells. Locally released NO diffuses to the smooth muscle cells in the proximity and activates the soluble guanylate cyclase to generate cGMP. The intracellular cGMP increase leads to muscle cell relaxation and dilatation of the blood vessel.

### DESCRIPTION OF THE INVENTION

The present invention provides a method for treating insufficient tissue perfusion with blood by the co-administration of an agent that increases cGMP synthesis and an agent that inhibits cGMP degradation. Such treatment leads to an increase in cGMP, which leads to an increase in endogenous nitric oxide synthase (NOS) activity and a consequent increase in nitric oxide in (endothelial or blood) cells, which leads to vasodilation.

More specifically, the method of the invention comprises the co-administration of any agent which increases nitric oxide (NO) generation in (endothelial or blood) cells (e.g. statins, bradykinin agonists, ACE-inhibitors (alone or combined with angiotensin receptor antagonists), estrogens, gene therapy with NOS) with an agent that provides the simultaneous and continuous inhibition of cGMP degradation in (vascular) smooth muscle cells (e.g. cGMP-phosphodiesterase inhibition by extended release formulations of dipyridamole or mopidamole). The co-administration of a statin and dipyridamole is a preferred embodiment of the invention.

Examples of agents which increase nitric oxide generation, and their appropropriate dosages are atorvastatin in oral daily doses of 5-80 mg, fluvastatin in oral daily doses of 10-40 mg, lovastatin in oral daily doses of 5-80 mg, pravastatin in oral daily doses of 5-40 mg or simvastatin in oral daily doses of 2.5-80 mg; captopril in oral daily doses of 12.5-150 mg, enalapril in oral daily doses of 2.5-40 mg, lisinopril in oral daily doses of 2.5-40 mg preindopril in oral daily doses of 1-8 mg or ramipril in oral daily doses of 1.25-20 mg (all these ACE inhibitors may be combined with angiotensin II receptor antagonists including candesartan (oral daily dose of 2-32 mg), irbesartan (oral daily dose of 75-300 mg), losartan (oral daily dose of 12.5-100 mg), telmisartan (oral daily dose of 20-160 mg) or valsartan (oral daily dose of 40-320 mg); micronized estradiol in oral daily doses of 1-30 mg, estradiol patches (daily dose 25-100 μg) or estradiol valerate 5-20 mg i.m. every 4 weeks.

The preferred agents for providing the inhibition of cGMP degradation in (vascular) smooth muscle cells are dipyridamole and mopidamole. An appropriate dosage would be 100-400 mg of dipyridamole or mopidamole per day.

As the simultaneous and continuous inhibition of cGMP degradation in (vascular) smooth muscle cells is desired it is preferred to employ an extended release formulation of dipyridamole or mopidamole, such as is described in U.S. Pat. No. 4,367,217, which is incorporated herein by reference.

An especially preferred agent for providing the simultaneous and continuous inhibition of cGMP degradation in (vascular) smooth muscle cells is an extended release formulation of dipyridamole and aspirin (acetyl salicylic acid), such as is described in U.S. Pat. No. 6,015,577, which is incorporated herein by reference.

What is claimed is:

1. A method for improving tissue perfusion with blood which comprises the coadministration of an agent that increases cGMP synthesis and an agent that inhibits cGMP degradation.

2. The method of claim 1 wherein the agent that increases cGMP synthesis is selected from the group consisting of statins, bradykinin agonists, ACE-inhibitors, estrogens, and gene therapy with NOS.

3. The method of claim 2 wherein the agent that increases cGMP synthesis is selected from the group consisting of:
(a) statins selected from the group consisting of atorvastatin, fluvastatin, lovastatin, pravastatin and simvastatin;
(b) ACE inhibitors selected from the group consisting of captopril, enalapril, lisinopril, preindopril and ramipril, optionally in combination with angiotensin II receptor antagonists selected from the group consisiting of candesartan, , irbesartan, losartan, telmisartan and valsartan; and,
(c) estradiol and estradiol valerate.

4. The method of claim 1 wherein the agent the agent that inhibits cGMP degradation is dipyridamole or mopidamole.

5. A method for improving tissue perfusion with blood which comprises the co-administration of a therapeutically effective amounts of a statin and dipyridamole or mopidamole.

6. The method of claims 4 or 5 wherein the dipyridamole or mopidamole is administered in a time-release formulation.
